(19) United States
(12) Patent Application Publication
(10) Pub. No.: US 2026/0306040 A1
(43) Pub. Date: Oct. 8, 2026

(54) SYSTEM AND METHOD FOR AN AUTOMATED MEASUREMENT OF OSSIFICATION OF AN ILIAC CREST APOPHYSES OF THE ILIUM OF A PELVIC BONE FOR THE ASSESSMENT OF RISSER STAGES

(71) Applicant: Siemens Healthineers AG, Forchheim (DE)

(72) Inventors: Andreas FIESELMANN, Erlangen (DE); Christian HUEMMER, Lichtenfels (DE); Christoph LUCKNER, Erlangen (DE)

(73) Assignee: Siemens Healthineers AG, Forchheim (DE)

(21) Appl. No.: 19/639,883

(22) Filed: Apr. 6, 2026

(30) Foreign Application Priority Data
Apr. 7, 2025 (DE) 10 2025 113 556.8

(51) Int. Cl. A61B 6/50; A61B 6/00

(52) U.S. Cl. CPC A61B 6/505; A61B 6/487; A61B 6/5205; A61B 6/5217

(57) ABSTRACT

One or more example embodiments relates to a system for a measurement of ossification of an iliac crest apophyses of an ilium of a pelvic bone for an assessment of Risser stages, the system comprising a data interface configured to receive a fluoroscopic image which depicts at least the ilium of the pelvic bone; a landmark model configured to recognize landmarks in the fluoroscopic image using machine learning, and the landmark model trained to identify at least one lateral starting point, one current ossification point and one medial end point of ossification as landmarks, wherein the system is configured to determine sections between at least one lateral starting point, one current ossification point and one medial end point of ossification, the sections represent distances at least from the starting point to the current ossification point and from the starting point or the current ossification point to the end point.

### CROSS-REFERENCE TO RELATED APPLICATION(S)

The present application claims priority under 35 U.S.C. § 119 to German Patent Application No. 10 2025 113 556.8, filed Apr. 7, 2025, the entire contents of which are incorporated herein by reference.

### FIELD

One or more example embodiments relates to a system and a method for the automated measurement of ossification of the iliac crest apophyses of the ilium of a pelvic bone for the assessment of Risser stages, as well as a medical system for X-ray imaging or diagnosis.

### RELATED ART

The progress of bone growth is a decisive factor in assessing the progression of scoliosis (cf. for example, “American College of Radiology (ACR) Appropriateness Criteria®, Scoliosis—Child”, https://acsearch.acr.org/docs/3101564/Narrative/). The more advanced the bone growth, the lower the likelihood of an increase in scoliosis. Bone growth usually ends between the ages of 16 and 20 and can be determined more accurately by measuring the ossification of the iliac crest apophyses of the ilium of a pelvic bone. During a child's growth phase, ossification of the iliac crest begins on the anterolateral side of the ilium and the ends on the posteromedial side. The iliac crest ossifies progressively from a starting point toward an end point (“iliac crest apophysis”). Ossification is complete when the iliac crest apophysis fuses, which is also a sign that bone growth has ended.

J.C. Risser showed in his work “The Iliac apophysis; an invaluable sign in the management of scoliosis” (cf. Clin Orthop Relat Res (2010) 468:646-653, https://pmc.ncbi.nlm.nih.gov/articles/PMC2816746/pdf/11999_2009_Article_1096.pdf, 1958) that the progression of scoliosis can be accurately assessed by observing the iliac crest apophysis. This led to the development of a system for classifying the progression of the iliac crest apophysis into so-called “Risser stages.”

The Risser stages describe ossification at the iliac crest and are an indication of the skeletal maturity of a child. The Risser stage is assessed based on an X-ray image of the pelvic bone and is extremely useful for the evaluation and treatment planning of adolescent idiopathic scoliosis.

The assessment of a Risser stage is based on a 6-point scale from 0 to 5. It should be noted that there are two staging systems (“Risser classification systems”), namely a US Risser classification system and a French Risser classification system, of which the US classification system is used more commonly. In both systems, Risser stage 0 means no ossification (yet) and stage 5 means complete iliac crest ossification and a fused iliac crest apophysis. The Risser stages of the two Risser classification systems are listed below:

US Risser stages:
0: No apophysis (ossification has not yet begun), 0% ossification;
1: Apophysis present, 0% to 25% ossification;
2: Apophysis present, 25% to 50% ossification;
3: Apophysis present, 50% to 75% ossification;
4: Apophysis present, 75% to 100% ossification;
5: No apophysis (apophysis fused), 100% ossification.

French Risser stages:
0: No apophysis (ossification has not yet begun), 0% ossification;
1: Apophysis present, 0% to 33% ossification;
2: Apophysis present, 33% to 67% ossification;
3: Apophysis present, 67% to 100% ossification;
4: Apophysis present, fusion has begun, 100% ossification;
5: No apophysis (apophysis fused), 100% ossification.

At present, Risser stages are assessed visually in everyday clinical practice. In general, physicians assess Risser stages without the use of special instruments (e.g., to measure the relative extent of ossification of the iliac crest). Visual assessment of Risser stages has several disadvantages. It is time-consuming, suffers from variability among different assessors, and requires experienced personnel.

As visual assessment has limitations, there are proposals to use AI-based methods for assessing Risser stages. Kaddioui et al. have proposed an approach for automated end-to-end Risser sign assessment using a convolutional neural network to categorize the Risser score on a 0-5 point scale. (cf. Kaddioui et al. (2020), Convolutional Neural Networks for Automatic Risser stage Assessment Radiology: Artificial Intelligence, 2:3,

https://pmc.ncbi.nlm.nih.gov/articles/PMC8082353/). Similarly, approaches have been proposed by Magnide et al. (cf. “Automatic bone maturity grading from EOS radiographs in Adolescent Idiopathic Scoliosis”, Computers in Biology and Medicine 136 (2021) 104681, https://doi.org/10.1016/j.compbiomed.2021.104681), Hwang et al. (cf. “Deep learning-based fully automatic Risser stage assessment model using abdominal radiographs”, Pediatric Radiology, 54:1692-1703, 2024; https://doi.org/10.1007/s00247-024-05999-1) and Xie et al. (cf. “Deep learning-based identification of spine growth potential on EOS radiographs”, Eur Radiol 34, 2849-2860, 2024; https://doi.org/10.1007/s00330-023-10308-9). They all use a trained neural network for the classification of Risser stages. These approaches are invariably very complicated and the derivation of Risser stages cannot be easily verified. Furthermore, none of these approaches follows the definitions in the US-American or French staging systems. They are therefore unable to provide the physician with explanations of their results when needed.

Hitherto, there has been no automatic solution which can reliably and transparently assess Risser stages.

### SUMMARY

One or more example embodiments provides a system and a method for the automated measurement of ossification of the iliac crest apophyses of the ilium of a pelvic bone for the assessment of Risser stages as well as a medical system for X-ray imaging or diagnosis, with which the disadvantages described above are avoided. For example, one or more example embodiments automatically determines Risser stages from images or at least to provide data which enables simple (and in particular unambiguous and reproducible) determination.

### BRIEF DESCRIPTION OF THE DRAWINGS

Exemplary embodiments are explained in more detail hereinafter with reference to the accompanying figures. In the various figures, identical components are provided with identical reference numbers. The figures are generally not to scale. They show:

FIG. 1 An approximate diagrammatic view of a CT system with a system according to one or more example embodiments,

FIG. 2 A fluoroscopic image of a hip bone with a suggested Risser classification system,

FIG. 3 A drawing of a hip bone with a suggested Risser classification system,

FIG. 4 Drawings of hip bones showing different Risser stages,

FIG. 5 A block diagram of a method according to one or more example embodiments,

FIG. 6 A fluoroscopic image with determined landmarks and sections,

FIG. 7 An alternative method of determining sections via an ellipse,

FIG. 8 Another alternative method of determining sections via straight lines,

FIG. 9 Another alternative method of determining sections to construct an angle.

### DETAILED DESCRIPTION

A system according to one or more example embodiments is used for the automated measurement of ossification of the iliac crest apophyses of the ilium of a pelvic bone for the assessment of Risser stages. It comprises the following components:
a data interface designed to receive a fluoroscopic image which depicts at least the ilium of a pelvic bone,
a landmark model capable of machine learning which is designed to recognize landmarks in fluoroscopic images and has been trained to identify as landmarks at least one lateral starting point, one current ossification point (the end point of ossification) and one medial end point of ossification. The system is designed to determine sections between these points, which represent a measure of distances at least from the starting point to the ossification point and from the ossification point to the end point.

The system also comprises a data interface designed to output sections.

The system is used for the automated measurement of ossification of the iliac crest apophyses of the ilium of a pelvic bone. A simple determination of Risser stages is already possible using the output sections. However, it is preferable if the system directly calculates and outputs Risser stages in an automated manner based on the sections. This is described in more detail hereinafter.

As the system requires images which show a pelvic bone or at least the iliac crest for its measurements, it includes the (first) data interface. This can be used for receiving and output, but different data interfaces can also be used for receiving and output. Such data interfaces are well known. A recorded fluoroscopic image can be received directly, or a fluoroscopic image can also be downloaded from a database.

A preferred fluoroscopic image is a radiographic image or a CT image. Bone structures, in particular an apophysis, should be clearly identifiable on fluoroscopic images. It may be assumed hereinafter that the fluoroscopic images are normal X-ray images or CT scans which may have been projected onto the frontal plane.

As the apophysis runs around the iliac crest, at least the ilium of a pelvic bone should be identifiable on the fluoroscopic image. It may be advantageous if the complete pelvic bone is visible on the fluoroscopic image as then the apophysis can be evaluated on both sides, making it possible to make particularly reliable statements.

Three points must be identified in this fluoroscopic image (where these exist). These are referred to as (anatomical) “landmarks”, as is customary in medicine. At least one lateral starting point and one medial end point of ossification are identified. In medicine, the starting point is determined by the Anterior Superior Iliac Spine (ASIS, for short) and the end point by the Posterior Superior Iliac Spine (PSIS, for short). The identification of the starting point and the end point is well known in medicine and is actually possible in every case, regardless of ossification. The lateral starting point can be defined anatomically as the projection of the anterior superior iliac spine (ASIS) onto the frontal plane, while the medial end point can be defined as the projection of the posterior superior iliac spine (PSIS) onto the frontal plane. If these two landmarks are difficult to identify in the image, other definitions at anatomically distinct points in the prior art may also be used. Furthermore, the starting point could also be defined as the start of ossification of the iliac crest actually visible in the fluoroscopic image.

The current ossification point does not necessarily have to be present (if ossification has not yet begun or has not yet been completed) and therefore does not always have to be identified. However, it much be searched for in the context of searching for landmarks. If an ossification point cannot be found, then (depending on the age of the patient) a Risser stage 0 or a Risser stage 5 can be assumed. If an ossification point cannot be identified and the patient is younger than 10 years old (ossification should have already begun at the age of ten), then Risser stage 0 can be assumed; if the patient is older than ten years of age, Risser stage 5 can be assumed.

The search for landmarks is performed by the (trained) landmark model capable of machine learning. This model has been trained to recognize landmarks, namely the aforementioned points in the fluoroscopic image. This means that their coordinates are determined in the fluoroscopic image or in a global coordinate system. Training can be carried out by providing a large number of (more than 100, preferably more than 1000 or even more than 10,000) fluoroscopic images as training images, in which the landmarks are marked as the starting point, the ossification point and the end point or their coordinates are specified as a basic truth. Fluoroscopic images which do not have any ossification points should also be used as training images. These can be marked with the additional classification “Risser stage 0” and “Risser stage 5” as a basic truth. However, in the case of the absence of ossification, the ossification point may be indicated at the position of the starting point, and in the case of completed ossification, the ossification point may be indicated at the position of the end point or medial to it. The position of the ossification point may later indicate a corresponding Risser stage. Other training methods are also conceivable, for example, segmenting the apophysis in fluoroscopic images and searching for points at the start (starting point) and the end (ossification point) of the apophysis, and determining an end point by segmenting the ilium.

According to one or more example embodiments, the system is designed to determine sections, in particular, section lengths, between the points. These sections must serve as a measure of the distances at least from the starting point to the ossification point and from the starting point or ossification point to the end point; however, the sections need not be straight.

One very straightforward possibility is that the sections run along the iliac crest or the apophysis, in other words, that they are curved. In this case, a (curved) section with a length A between the starting point and the ossification point can be determined, and a section with a length B between the starting point and the end point. Basically, these two sections are sufficient to easily determine a Risser stage. The quotient Q=A/B is calculated, and the result is plotted in a Risser classification system to determine its range of values. Alternatively, it is also possible to determine a section with a length C between the ossification point and the end point. The quotient would then be calculated as Q=A/(A+C), as A+C=B.

It is also possible to determine other sections as well as, rather than directly from sections, to derive Risser stages from angles which are determined using these sections. This will be described in more detail below.

The determined sections are then output via a data interface. The sections can be output directly, for example as mathematical expressions, or for example as vectors, functions or splines. However, the section lengths can also be output directly. This output can be provided to a user, but it is preferable that this output is provided to an apparatus which determines Risser stages in an automated manner. This can simply be a computing apparatus which calculates the quotient and classifies the result in a Risser classification system.

A method according to one or more example embodiments is used for the automated measurement of ossification of the iliac crest apophyses of the ilium of a pelvic bone to assess Risser stages. It is preferably performed using a system according to one or more example embodiments. The (in particular, computer-aided) method comprises the following steps:
Provision of a fluoroscopic image which depicts at least the ilium of a pelvic bone,
Identification of landmarks in the fluoroscopic image, at least one lateral starting point, one current ossification point and one medial end point of ossification being sought as landmarks in the fluoroscopic image,
Determination of sections between these points which represent a measure of distances at least from the starting point to the ossification point, and from the starting point or ossification point to the end point,
Determination of a predominant Risser stage on the fluoroscopic image based on the sections (preferably the section lengths),
Outputting of the determined Risser stage.

Essentially, the method has already been described above in conjunction with the system. Based on the determined sections, a Risser stage can be easily identified by examining the section lengths or by examining the angles between the sections. As already described above, the (in this case curved) sections can lie on the iliac crest or on the iliac crest apophysis, and their lengths can be divided into each other. However, an arched curve can also be constructed between the starting point and the end point which passes through the ossification point, for example, a spline or the arc of an ellipse, and section lengths on this curve can be considered and their quotient calculated. Alternatively, a straight section between the starting point and the end point can be considered and a straight line perpendicular to it, which passes through the ossification point, can be drawn. On this straight section, the section length B between the starting point and the end point, and the length A between the starting point and the intersection of the straight line are then determined, and their quotient calculated (Q=A/B). In this case, however, the value Q would need to be adjusted, or a modified Risser classification system would need to be used as, even though a clear conclusion is possible, the Risser stages lie on a curve, whereas here a straight line is being considered. However, it is also possible to determine the midpoint of the straight section between the starting point and the end point and to draw a straight line which passes through this midpoint and the ossification point. The Risser stage can then be clearly determined from the angle between the straight line and the straight section. However, in this case as well, it is advantageous to use a suitably adapted Risser classification system (with angle intervals) or to convert the determined angle into a radian measure on a predetermined curve.

In practice, a preferred method can be carried out as follows:

First of all, as an optional step, a fluoroscopic image provided can be examined according to Risser stages 0 (no ossification) and 5 (completed ossification with fusion) (and, if necessary, also according to other Risser stages, with which a comparison can be made at the end). No ossification points will be found in these images, and a result could be generated quickly and easily using methods already known. The Risser stage can be determined using a trained neural network in the prior art (see above). If Risser stage 0 or 5 has been identified, following the subsequent steps is no longer necessary (i.e., essentially the steps of the method described above) as these are advantageous for the other Risser stages 1 to 4 (and no ossification point may be found). However, the method according to one or more example embodiments may still be followed to verify the result.

However, if ossification is underway, the starting point, the ossification point and the end point are identified as landmarks in the fluoroscopic image (possibly after this step). Their coordinates are preferably specified. The landmark model described above can be used for this purpose. If the ossification point is not found, ossification is either complete or it has not yet begun. To this end, the age of the patient can be determined as described above, and a Risser stage 0 or 5 derived therefrom. It should be noted that in the event of complete ossification without complete fusion, the ossification point can still be detected, namely ideally at the position of the end point.

Once the points and their coordinates have been identified, the sections are determined. Preferably, the arc length on the iliac crest is determined from the starting point to the ossification point and from the starting point or ossification point to the end point. In this regard, however, other approaches are also possible, as already described previously. In practice, for example, a trained neural measurement model can be used to segment the iliac crest and the curved sections, or their lengths can be derived directly from the segmented iliac crest. A test should be performed here as to whether the ossification point lies within a specified area in the vicinity of the iliac crest. To do this, an area around the iliac crest can be defined and checked as to whether the position of the ossification point lies within this area. Alternatively, however, a mathematical curve can be used, or other methods can be applied (see above). In addition, it could be verified that the ossification point is neither located medially from the end point nor laterally to the starting point.

From these sections, the ossified portion can then be determined (using the aforementioned quotient or angles) or a Risser stage can be directly derived (using an appropriate Risser classification system). It should be noted that in the event that the French Risser classification system is used, the onset of fusion should still be verified in the case of 100% ossification in order to distinguish between Risser stages 3, 4 and 5.

It is preferable to then compare the Risser stage obtained in this manner with a Risser stage which was obtained using a method according to the prior art. This provides additional certainty.

It is also preferable that both iliac bones of the patient are examined, which provides additional certainty. The results for each iliac bone can be provided separately from each other. However, the results for both sides of the pelvic bone can also be combined, for example, in the form of a maximum or an average value.

When the results are displayed, the fluoroscopic image can also be displayed, and in particular, the boundaries of the Risser stages indicated in the form of reference lines. This makes a report clearer and more concise. The method can be applied to multiple longitudinal examinations of the same patient. In this case, the same landmarks on the ilium can be used, and the prior information that the ossification point moves from the lateral to the medial side can serve as a plausibility check for the results.

A medical system according to one or more example embodiments is preferably a diagnostic system, a CT system or another X-ray system, and is used for X-ray imaging or diagnosis. It comprises a system according to one or more example embodiments or is designed to perform a method according to one or more example embodiments.

One or more example embodiments may, for example, be implemented in the form of a computer unit with suitable software. For this purpose, the computer unit may, for example, have one or more microprocessors or the like working together. In particular, one or more example embodiments may be implemented in the form of suitable software program modules in the computer unit. The advantage of a broadly software-based implementation is that even computer units already in use can be easily retrofitted via a software update or a firmware update in order to be operated in the manner according to one or more example embodiments. In this respect, the object is also achieved in that a corresponding computer program product with a computer program can be loaded directly into a storage facility of a computer unit, with program sections for executing all the steps of the method according to one or more example embodiments when the program is executed in the computer unit. In addition to the computer program, such a computer program product may, where applicable, comprise additional components such as, for example, documentation and/or additional components, as well as hardware components such as, for example, hardware keys (dongles, etc.) for use of the software.

A computer-readable medium, for example, a memory stick, a hard drive, or any other portable or built-in data carrier on which the program sections of the computer program which are readable and executable by a computer unit are stored may be used to transport data to the computer unit and/or to store it on or in the computer unit.

Further, particularly advantageous embodiments and developments of the invention will emerge from the dependent claims and the following description, it being possible for claims in one claim category to also be developed analogously to the claims and descriptions of another claim category and it being possible, in particular, for individual features of different exemplary embodiments or variants to also be combined to form new exemplary embodiments or variants.

A preferred system is used to assess Risser stages. In addition, it is designed to determine the predominant Risser stage in a fluoroscopic image based on the sections, in particular, the section lengths derived therefrom. This has already been described above and preferably relates to the formation of a quotient of the section to the ossification point by the entire section between the starting point and the end point. It is preferable that the system is designed to divide a section A from the starting point to the current ossification point by an entire section B from the starting point to the end point and to classify the value obtained as a result (Q=A/B) in a specified Risser classification system (US or French), and to determine a Risser stage therefrom. If, for example, Q is 0.4 for arched sections along the iliac crest Q, then a Risser stage 2 can be assumed. Alternatively, it is preferable that the system is designed to determine an angle between a straight section M from the starting point to the end point, and a section N from the midpoint of section M to the ossification point, and to determine a Risser stage from this angle.

In the event of an absence of ossification of the iliac crest apophyses, the system is preferably designed to place the ossification point at the starting point, to assign the section between the starting point and the ossification point the length zero or to directly assign the Risser stage 0. This makes the statement about Risser stage 0 very simple.

Alternatively or in addition, in the event that fusion of the iliac crest apophyses with the ilium has been completed, the system is preferably designed to place the ossification point at the end point or medial to it, to equate the section between the starting point and the ossification point with the section between the starting point and the end point or to define this section as greater, or to directly assign a Risser stage 5. It should be noted here that, in practice, the ossification point may be medial to the end point without Risser stage 5 being present. Therefore, an ossification point medial to the end point should preferably not be solely relied on for a Risser stage 5.

It is preferable that the landmark model has been trained using training data in the form of fluoroscopic images of the ilium, which show a lack of ossification of the iliac crest apophyses and, as a basic truth, specify the position of the ossification point at the position of the starting point or indicate a Risser stage 0, and/or show complete fusion of the iliac crest apophyses with the ilium and, as a basic truth, indicate the position of the ossification point at the position of the end point or indicate a Risser stage 5. It should be noted here that if the output node of the landmark model only “knows” points, training is only performed on points, and if the output node also includes Risser stages, these can also be output directly.

A preferred system is characterized in that the system comprises a measurement model capable of machine learning which has been trained to segment the iliac crest. Segmentation of the iliac crest is known in the prior art and can take place, for example, using training images in which the iliac crest is marked as a basic truth. The system, preferably the measurement model, is then designed to detect the edge of the iliac crest and to determine lines on the edge of the iliac crest between two points as sections between the starting point, the ossification point and the end point. Particularly preferably, the system, preferably the measurement model, is designed to determine the lengths of the sections. It is preferable that the system, in particular, the measurement model, is designed to verify whether the ossification point lies on the iliac crest. It should be noted here that the statement “on the iliac crest” refers to the customary area of ossification.

A preferred system is designed to construct a mathematically defined curved structure on which the starting point, the ossification point, and the end point lie. This curved structure is preferably an ellipse (or its arc) or a spline. This curved structure can be used as an alternative to a segmented iliac crest as the arc essentially follows the course of the iliac crest. The system is further designed to determine curved sections between the starting point, the ossification point and the end point in the form of lines on the curved structure between two points. Furthermore, the system is designed to determine Risser stages based on this curved structure.

With regard to the method, it is accordingly preferable that a mathematically defined curved structure be constructed on which the starting point, the ossification point and the end point lie, and that curved sections in the form of lines on the curved structure between two points be determined as sections between the starting point, the ossification point and the end point, and that Risser stages be determined based on this curved structure. This curved structure is preferably an ellipse or a spline.

It is preferable that the curved structure is an ellipse, the major half-axis of which spans the starting point and the end point, and that the system is designed to determine curved sections in the form of lines on the ellipse between two points as sections between the starting point, the ossification point and the end point. This has the advantage that segmentation of the iliac crest is not necessary. Alternatively, the system is designed to project the ossification point along a direction of a minor half-axis of the ellipse onto the major half-axis and to determine lines along the major half-axis of the ellipse as sections between the starting point, the ossification point and the end point. This has the additional advantage that section lengths can be calculated very easily; however, to determine a Risser stage, the result should then be adjusted, or a correspondingly adjusted Risser classification system should be used. Alternatively, the system is designed to determine the point of intersection of the minor half-axis with the major half-axis and to determine an angle between the major half-axis and a section between the point of intersection and the ossification point. This has the advantage that in addition to simple calculation, clear visual representation is also possible. It is particularly preferable that, for reasons of plausibility, the system is designed to verify that the ossification point is not lateral to the starting point and not medial to the end point.

Accordingly, in the method it is preferable that an ellipse be constructed as the curved structure, with its major half-axis spanning the starting point and the end point. Curved sections in the form of lines on the ellipse between two points are then preferably determined as sections between the starting point, the ossification point and the end point. Alternatively, it is preferable that the ossification point be projected onto the major half-axis along a direction of a minor half-axis of the ellipse, and that lines along the major half-axis of the ellipse between two points be determined as sections between the starting point, the ossification point and the end point. Alternatively, it is preferable that the point of intersection of the minor half-axis with the major half-axis be determined and that an angle between the major half-axis and a section between the point of intersection and the ossification point be determined.

A preferred system comprises a Risser model which is designed to classify a Risser stage in the fluoroscopic image, preferably from all possible Risser stages 1 to 5 according to a specified Risser classification system (French or US). Such Risser models are known in the prior art. The system is preferably designed to compare the Risser stage from the Risser model identified in a fluoroscopic image with the Risser stage identified in this fluoroscopic image based on the determined sections. It is preferable that the Risser model has been trained using fluoroscopic images in which Risser stages were specified as a basic truth.

It is preferable that the method is performed on both iliac bones, or that the system is designed to specify sections (or Risser stages) for both sides of a pelvic bone. This improves the result. A classification using the entire image as input can assess skeletal maturity (which is, in principle, reflected by the Risser stage) even more comprehensively. It should be noted here that skeletal maturity can also be estimated, for example, based on the femoral heads. This can be used, in particular, to verify the consistency of the result and, in the event that different results are available for the skeletal maturity of a patient, a warning could be displayed, or a maximum or average value could be reported.

A preferred system is characterized in that the landmark model and/or the Risser model and/or the measurement model comprises a convolutional network, preferably a Convolutional Neural Network and/or a U-Net.

In an embodiment of the method, it is preferable that the section A from the starting point to the current ossification point is divided by the entire section B from the starting point to the end point, and the value obtained as a result is classified in a predetermined Risser stage (in particular, US or French) in order to determine a Risser stage therefrom. It is preferable that value intervals are specified, numbered at least from 1 to 4, that the value interval in which the obtained quotient lies is determined, and that the number of the value interval is output as the Risser stage. In the US Risser classification system, these intervals would range from 0 to 25% (Risser stage 1), from 25% to 50% (Risser stage 2), from 50% to 75% (Risser stage 3) and from 75% to 100% (Risser stage 4).

In one embodiment of the method, if the ossification point could not be found, it is preferable to retrieve data regarding the age of the person from whom the fluoroscopic image was obtained from the time the image was captured, and if the age is below a predetermined threshold (for example, 10 years), Risser stage 0 is output without determining the sections, and if the age is above this predetermined threshold, Risser stage 5 is output without determining the sections.

It is preferable to apply the method according to one or more example embodiments to a patient multiple times over a specified period or until Risser stage 5 is reached. This results in a good assessment of the growth trajectory of the patient, and thus the progression of scoliosis can be very well documented and correlated with growth.

Preferably the method also comprises the stages:
Determination of a Risser stage in the fluoroscopic image in addition to section-based determination (in the conventional manner), preferably via automated image processing (for example, a Risser model as mentioned above), in particular using a system according to one or more example embodiments,
Comparison of the resulting Risser stage identified in the fluoroscopic image with the Risser stage identified in this fluoroscopic image based on the determined sections.

It is preferable that a warning be issued if the Risser stages do not match. For example, the relevant anatomical areas which have influenced the determination of the Risser classification by the classification network could be displayed using color coding.

It is preferable that in the event that Risser stage 0 or Risser stage 5 has been identified in the additional determination of a Risser stage, no section-based determination of the Risser stage and no comparison takes place.

The use of AI-based methods (AI: Artificial Intelligence) is preferable for the method according to one or more example embodiments. Artificial Intelligence is based on the principle of machine learning and as a rule, it is implemented using an adaptive algorithm which has been trained accordingly. The term “machine learning” is used to describe this method, the principle of “deep learning” also being included here.

Preferably, components of one or more example embodiments are available as a “cloud service”. Such a cloud service is used to process data, in particular, via artificial intelligence, but may also be a service based on customary algorithms or a service in which evaluation by humans takes place in the background. Generally speaking, a cloud service (hereinafter also referred to as “cloud”) is an IT infrastructure in which, for example, storage space or computing power and/or application software are made available via a network. Communication between the user and the cloud takes place via data interfaces and/or data transmission protocols. In the present case, it is particularly preferable that the cloud service provides both computing power and application software.

In the context of a preferred method, data which is obtained as part of one or more example embodiments is transmitted via the network to the cloud service. This service comprises a computing system which does not include the local computer of the user as a rule. The method can be implemented using a set of commands in a network. The data processed in the cloud is subsequently sent back to the local computer of the user via the network.

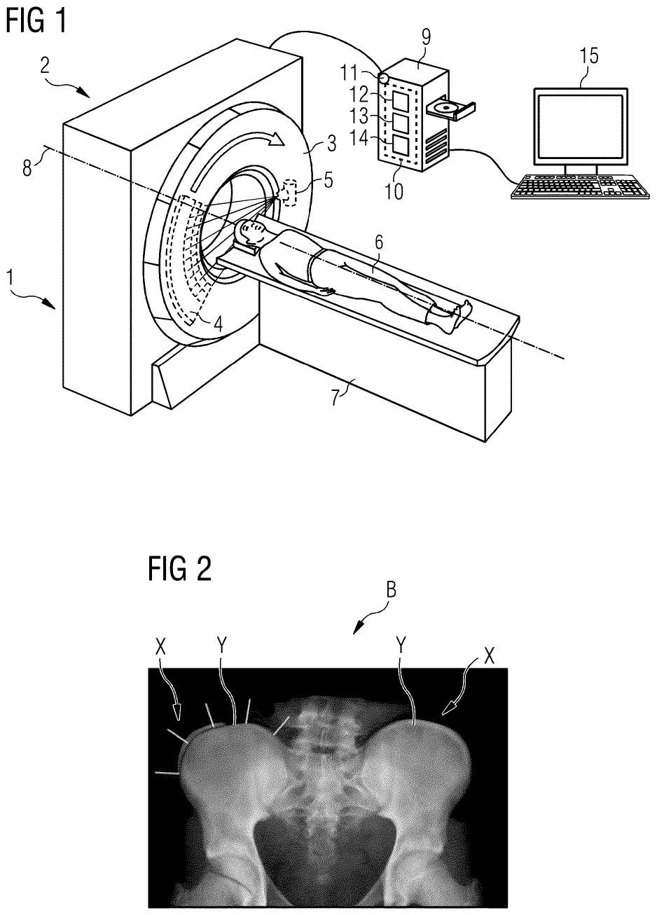

FIG. 1 shows a CT system 1 as an example of a medical system 1 with a radiation detector 4 and an X-ray source 5. The X-ray source 5 is designed to expose the radiation detector 4 to X-rays. The CT system 1 shown comprises a gantry 2 with a rotor 3. The rotor 3 comprises the X-ray source 5 and the radiation detector 4, which is designed to detect X-rays. In practice, the images can also be captured using a standard X-ray device.

The rotor 3 is rotatable about the axis of rotation 8. A patient 6 is positioned on the patient table 7 and can be moved along the axis of rotation 8 by the gantry 2. The control facility 9 is provided to control the CT system 1 and/or to generate an image dataset based on signals detected by the radiation detector 4.

A (raw) X-ray image dataset of the patient 6 is customarily acquired from a multiplicity of angle directions via the radiation detector 4. Subsequently, based on the (raw) X-ray image dataset, an image dataset can be reconstructed using a mathematical method, for example, comprising a filtered back projection or an iterative reconstruction method.

The control facility 9 is used here to control the CT system 1 and to analyze its images. An input facility with a display 15 on which results can be displayed is connected to this computing unit 9.

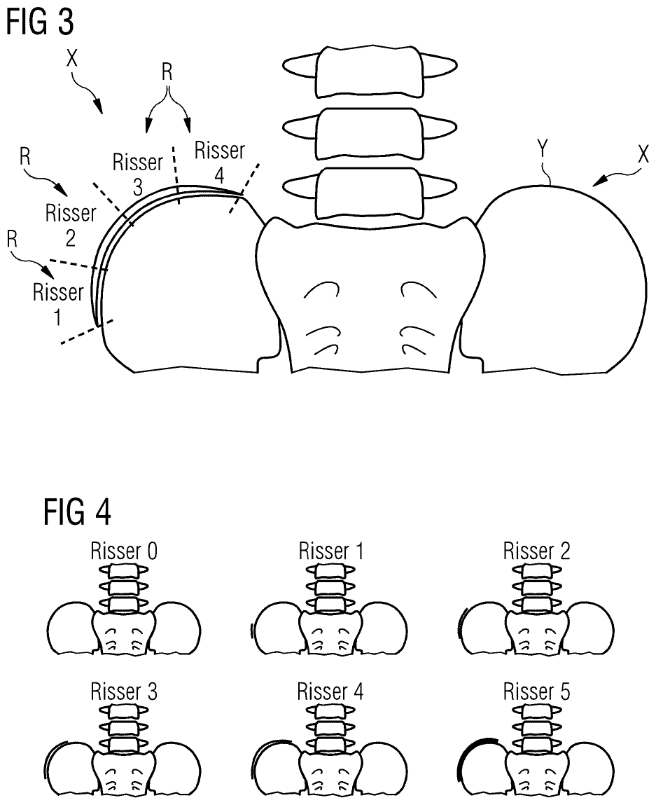

The control facility 9 comprises a system according to one or more example embodiments 10 for the automated measurement of ossification of the iliac crest apophyses Y of the ilium X of a pelvic bone (cf., for example, FIG. 2 or 3) for the assessment of Risser stages R; the system 10 comprises a data interface 11, a landmark model 12, a measurement model 13, and a Risser model 14.

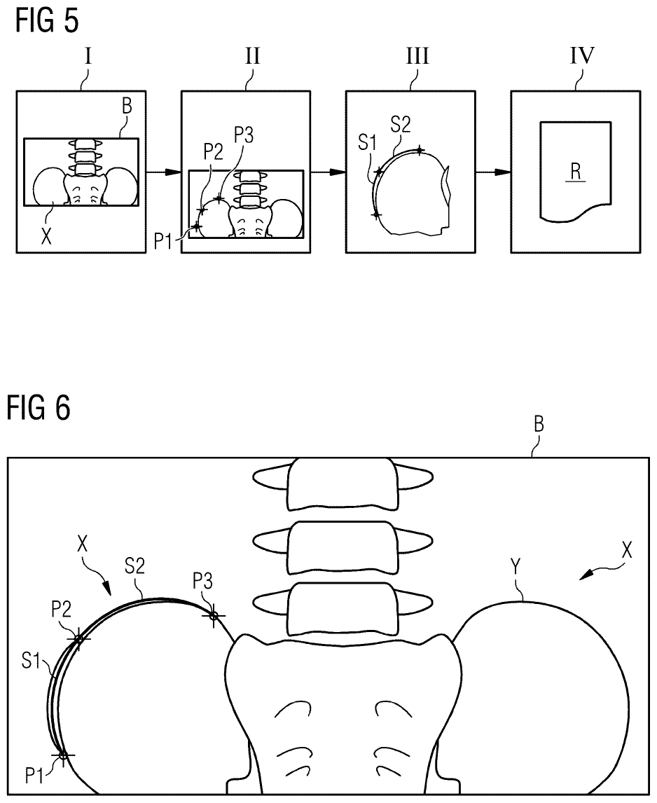

The data interface 11 is used here to receive a fluoroscopic image B from the image detector 4, which has been reconstructed, and to output results regarding Risser stages R on the display 15. For the specific mode of operation, which is explained in more detail hereinafter, please refer to FIG. 4, but also to FIGS. 5 to 9.

The landmark model 12 capable of machine learning is used to detect landmarks in the fluoroscopic image B. It has been trained to identify as landmarks at least one lateral starting point P1, one current ossification point P2 and one medial end point P3 of ossification.

The system 10 is designed to determine sections S1 and S2 between these points P1, P2, P3, which serve as a measure of the distances at least from the starting point P1 to the ossification point P2, and from the starting point P1 or the ossification point P2 to the end point P3. This is achieved using the measurement model 13 capable of machine learning, which has been trained to segment the iliac crest Y. In this case, the measurement model is also designed to detect the edge of the iliac crest Y and to determine lines on the edge of the iliac crest Y between two points P1, P2, P3 as sections S1, S2 between the starting point P1, the ossification point P2 and the end point P3 (cf. FIG. 5).

Using these sections, a quotient can be calculated very easily, and the Risser classification system can be read directly therefrom. To do this, a section A S1 from the starting point P1 to the current ossification point P2 is divided by an entire section B S2 from the starting point P1 to the end point P3, and the value obtained in this manner is classified in a specified Risser classification system. From this, a Risser stage R is then determined.

The Risser model 14 is designed to classify a Risser stage R in the fluoroscopic image B. This can be done in the conventional manner. The system 10 is designed to compare the Risser stage R identified in the fluoroscopic image B from the Risser model 14 with the Risser stage R identified in this fluoroscopic image B, based on the determined sections S1, S2. An average value can then be calculated, or warnings issued in the event of deviations.

FIG. 2 shows a fluoroscopic image B of a hip bone with an ilium X on the right and left. On the ilium X on the left in the image, a US Risser classification system is indicated by lines on the iliac crest Y.

FIG. 3 shows a drawing of a hip bone with a suggested US Risser classification system on the ilium X shown on the left. The ossification of the iliac crest Y is complete here, but fusion has not yet begun, so this represents Risser stage 4 R.

FIG. 4 shows drawings of hip bones at different Risser stages R. Starting at the top left (Risser 0), ossification starts in the middle upper image (Risser 1) and continues to the right (Risser 2). In the bottom row, the apophysis continues to develop from left to right until it ends in the bottom right image with its fusion with the iliac crest Y (Risser 5).

FIG. 5 shows a block diagram of a method according to one or more example embodiments for the automated measurement of ossification of the iliac crest apophyses of the ilium Y of a pelvic bone for the assessment of Risser stages R (cf. for example, FIG. 4).

In step I, a fluoroscopic image B is provided, which shows at least the ilium X of a pelvic bone.

In step II, landmarks are detected in the fluoroscopic image B, at least one lateral starting point P1, one current ossification point P2 and one medial end point P3 of ossification being sought (and also found here) as landmarks in the fluoroscopic image B.

In step III, sections S1 and S2 are determined between these points P1, P2, P3, which represent a measure of the distances at least from the starting point P1 to the ossification point P2 and from the starting point P1 and/or the ossification point P2 to the end point P3.

In step IV, the dominant Risser stage R in the fluoroscopic image B is determined based on the sections S1, S2. The determined Risser stage R can then be output.

FIG. 6 shows a fluoroscopic image B with determined landmarks P1, P2, P3 and sections S1, S2. In this example, the sections S1 and S2 lie on the iliac crest Y and on the apophysis, respectively, and are curved lines. The Risser stage R can be determined very easily from the ratio of these sections S1 and S2.

However, this is not the only way of determining Risser stages R. Instead of identifying the iliac crest Y or the apophysis, a mathematically defined curved structure can also be constructed on which the starting point P1, the ossification point P2 and the end point P3 lie. Then curved sections S1 and S2 between the starting point P1, the ossification point P2 and the end point P3 can be determined in the form of lines as sections S1, S2 on the curved structure between two points, and Risser stages R can be determined based on this curved structure.

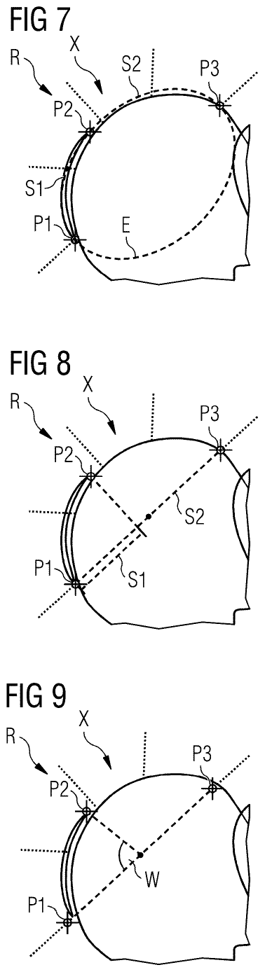

FIG. 7 shows an alternative method for determining sections S1 and S2. Here, the curved structure is an ellipse E, the major half-axis of which spans the starting point P1 and the end point P3. Here, the curved sections S1, S2 between the starting point P1, the ossification point P2 and the end point P3 are determined as sections S1, S2 in the form of lines on the ellipse E between two points. A Risser stage R can also be easily determined from their ratio.

FIG. 8 shows another alternative for determining the sections S1, S2 via straight lines.

Here, a straight line is drawn between the starting point P1 and the end point P3 (which essentially corresponds to the major half-axis of the ellipse E in FIG. 5), and the ossification point P2 is projected onto this line along a direction orthogonal to the line (which essentially corresponds to the minor half-axis of the ellipse E in FIG. 5). As shown here, a straight line which runs orthogonally to the line through the ossification point 2 can also be drawn. Lines along the line between the starting point P1 and the end point P3 are now considered as sections S1, S2 between the starting point P1, the ossification point P2 and the end point P3 (to the end point P3 and to the projection of the ossification point P2). As can be seen in the dotted Risser classification system, the ratio of sections S1 and S2 is a clear measure of the Risser stage R.

FIG. 9 shows another alternative for determining sections S1 and S2 to construct an angle W. As in FIG. 6, a straight line is drawn between the starting point P1 and the end point P3. The midpoint of this line is now determined, and a straight line is drawn through this midpoint and the ossification point P2. As can be seen in the dotted Risser classification system, the angle W of section S1 between the midpoint and the ossification point P2 on the straight line to the line S2 is a clear measure of the Risser stage R.

Finally, it should be noted once again that the invention described in detail above only concerns exemplary embodiments which may be modified in various ways by a person skilled in the art without departing from the scope of the invention. Furthermore, the use of the indefinite article “a” or “an” does not preclude the possibility that the relevant features may also be present multiple times. Likewise, terms such as “unit” do not preclude the possibility that the relevant components may consist of multiple interacting subcomponents which may also be spatially distributed. The term “a number” is to be understood as meaning “at least one”. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility (also referred to as an data processing system) or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A system for an automated measurement of ossification of an iliac crest apophyses of an ilium of a pelvic bone for an assessment of Risser stages, the system comprising:
a data interface configured to receive a fluoroscopic image which depicts at least the ilium of the pelvic bone;
a landmark model configured to recognize landmarks in the fluoroscopic image using machine learning, and the landmark model trained to identify at least one lateral starting point, one current ossification point and one medial end point of ossification as landmarks, wherein
the system is configured to determine sections between at least one lateral starting point, one current ossification point and one medial end point of ossification, the sections represent distances at least from the starting point to the current ossification point and from the starting point or the current ossification point to the end point; and
a data interface configured to output the sections.

2. The system of claim 1, wherein the system is configured to
divide a first section from the starting point to the current ossification point by an entire section from the starting point to the end point to generate a first value and to classify the first value in a predefined Risser classification system, and to determine a Risser stage based on classifying the first value, or
determine an angle between a straight section from the starting point to the end point and a second section from a midpoint of the straight section to the current ossification point, and to determine a Risser stage from the angle between the straight section and the section.

3. The system of claim 1, wherein at least one of
in an absence of ossification of the iliac crest apophyses, the system is configured to place the current ossification point at the starting point, to assign the section between the starting point and the current ossification point a length of zero or to assign a Risser stage 0, or
in a complete fusion of the iliac crest apophyses with the ilium, the system is configured to place the current ossification point at the end point or medial to the end point, to set the section between the starting point and the current ossification point to be equal to or greater than the section between the starting point and the end point or to assign a Risser stage 5.

4. The system of claim 1, further comprising:
a measurement model configured to segment the iliac crest using machine learning, and the system is configured to detect an edge of the iliac crest and to determine lines on the edge of the iliac crest between two points as sections between the starting point, the current ossification point and the end point.

5. The system of claim 1, wherein the system is configured to
construct a mathematically defined curved structure, on which the starting point, the current ossification point and the end point lie,
determine curved sections as sections between the starting point, the current ossification point and the end point in the form of lines on the curved structure between two points, and
determine Risser stages based on the curved structure.

6. The system of claim 1, further comprising:
a Risser model configured to classify a Risser stage in a fluoroscopic image, wherein the system is configured to compare the Risser stage detected in the fluoroscopic image from the Risser model with the Risser stage detected in the fluoroscopic image based on the determined sections, wherein the Risser model has been trained using training fluoroscopic images in which Risser stages were specified as a basic truth.

7. The system of claim 6, wherein at least one of the landmark model, the Risser model, or the measurement model comprise a convolutional network.

8. A method for an automated measurement of ossification of an iliac crest apophyses of an ilium of a pelvic bone for an assessment of Risser stages, the method comprising:
providing a fluoroscopic image which depicts at least the ilium of the pelvic bone;
identifying landmarks in the fluoroscopic image, wherein at least one lateral starting point, one current ossification point and one medial end point of the ossification are sought as landmarks in the fluoroscopic image;
determining sections between these points, which represent a distances at least from the starting point to the ossification point and from the starting point or the ossification point to the end point;
determining a Risser stage predominant in the fluoroscopic image based on the sections; and
outputting of the determined Risser stage.

9. The method of claim 8, wherein a first section from the starting point to the current ossification point is divided by an entire section from the starting point to the end point to generate a first value, and the first value is classified in a predetermined Risser classification system to determine a Risser stage.

10. The method of claim 8, wherein
a mathematically defined curved structure is constructed on which the starting point, the current ossification point and the end point lie, and
curved sections are determined as sections between the starting point, the current ossification point and the end point in the form of lines on the curved structure between two points, and
Risser stages are determined based on the curved structure.

11. The method of claim 8, wherein, if the current ossification point is not found, data regarding an age of a person from whom the fluoroscopic image was obtained at a time of acquisition is retrieved, and if the age is below a predetermined threshold value, Risser stage 0 is output without determining sections, and if the age is above the predetermined threshold value, Risser stage 5 is output without determining sections.

12. The method of claim 8, further comprising:
determining a Risser stage in the fluoroscopic image via automated image processing;
comparing the Risser stage identified from the automated image processing with the Risser stage identified in the fluoroscopic image based on the determined sections.

13. A medical system for X-ray imaging or diagnosis, comprising:
the system of claim 1.

14. A non-transitory computer program product comprising commands, when executed by a computer, cause the computer to perform the method of claim 8.

15. A non-transitory computer-readable storage medium comprising commands, when executed by a computer, cause the computer to perform the method of claim 8.

16. The system of claim 3, wherein the landmark model has been trained by providing training data in the form of fluoroscopic images of the ilium which at least one of
show an absence of ossification of the iliac crest apophyses and, as a basic truth, indicate a position of the current ossification point at the position of the starting point or indicate a Risser stage 0, or
show a complete fusion of the iliac crest apophyses with the ilium and, as a basic truth, indicate the position of the current ossification point at a location of the end point or indicate a Risser stage 5.

17. The system of claim 4, wherein the system is configured to verify whether the current ossification point lies on the iliac crest.

18. The system of claim 5, wherein the curved structure is an ellipse, a major half-axis of which spans the starting point and the end point, and the system is configured to
determine the curved sections as sections between the starting point, the current ossification point and the end point in the form of lines on the ellipse between two points,
project the current ossification point along a direction of a minor half-axis of the ellipse onto the major half-axis, and to determine lines as sections between the starting point, the current ossification point and the end point along the major half-axis of the ellipse, or
determine the point of intersection of the minor half-axis with the major half-axis and to determine an angle between the major half-axis and a section between the point of intersection and the ossification point, wherein
the system is configured to verify that the current ossification point is not lateral to the starting point and not medial to the end point.

19. The method of claim 9, wherein value intervals are specified, numbered at least from 1 to 4, a value interval in which an obtained quotient lies is determined and the number of the value interval is output as the Risser stage.

20. The method of claim 10, wherein an ellipse is constructed as the curved structure, a major half-axis of which spans the starting point and the end point, and
curved sections are determined as sections between the starting point, the current ossification point and the end point in the form of lines on the ellipse between two points,
the current ossification point is projected onto the major half-axis along a direction of a minor half-axis of the ellipse, and lines are determined as sections between the starting point, the current ossification point and the end point along the major half-axis of the ellipse between two points, or
the point of intersection of the minor half-axis with the major half-axis is determined, and an angle between the major half-axis and a section between the point of intersection and the current ossification point is determined.
